(19) United States
(12) Patent Application Publication
(10) Pub. No.: US 2009/0285865 A1
(43) Pub. Date: Nov. 19, 2009

(54) Palatable, solid oral formulations for male chemical sterilization

(76) Inventor: Shalaby W. Shalaby, Anderson, SC (US)

Correspondence Address: LEIGH P. GREGORY, PO BOX 168, CLEMSON, SC, 29633-0168, US

(21) Appl. No.: 12/454,351

(22) Filed: May 14, 2009

Related U.S. Application Data
(60) Provisional application No. 61/127,625, filed on May 14, 2008.

(51) Int. Cl. A61K 9/00; A61K 33/30; A61K 31/315; A61K 31/4164; A61K 31/167; A61K 31/496; A61K 31/12; A61K 31/122

(52) U.S. Cl. 424/400; 424/643; 514/494; 514/399; 514/629; 514/254.07; 514/681; 514/689

(57) ABSTRACT

Palatable, solid oral formulations or compositions include at least one bioactive agent capable of chemical sterilization of human and animal males. Depending on the targeted species, the bioactive formulations comprise suitable additives, excipients, water-swellable compositions, and/or processing aids and can be made into uncoated or coated forms to modulate the release profile of the bioactive agents. Among the bioactive agents are zinc compounds and complexes and organic drugs with established pharmacological activities, which are traditionally unrelated to any form of contraception.

The present application claims the benefit of prior provisional application U.S. Ser. No. 61/127,625, filed May 14, 2008.

### FIELD OF THE INVENTION

The present invention is directed to ingestible compositions intended to cause chemical sterility of human and animal males as a practical, economical means to control population. In effect, this invention is directed toward bioactive, edible compositions formulated as palatable, oral formulations for ingestion by human and animal males. Included in the latter are rodents, especially rats, beavers, and squirrels, to render them infertile, temporarily or permanently, leading to the reduction in their populations through chemical sterilization. The palatable formulations are designed to be self-standing, chewable or swellable solids or can be mixed or added to food ingredients. The bioactive agents, or combinations thereof, are selected from among those closely or remotely related to animal contraception as well as those known for totally unrelated biological effects.

### BACKGROUND OF THE INVENTION

Condoms and vasectomy are the only fertility control methods available for male humans. Over fifty million surgical vasectomies have been performed worldwide. However, the widespread use of vasectomy is limited due mainly to fear of genital operations. On the other hand, chemical sterilization offers an alternative to surgery. Meanwhile, it has been acknowledged that androgen suppression in men and male animals can be an approach to immobilizing sperms and inhibiting fertilization as a means to achieving male contraception that is occasionally referred to as chemical castration. Among those used for humans include injectable, controlled release formulations containing high cost synthetic peptides, which are intended, primarily, for treating prostatic cancer with temporary castration as a side effect. However, the prior art is silent as to the use of oral, controlled drug release formulations intended, primarily, for male contraception or temporary castration that can be extended over long periods of time through continued ingestion of said formulations containing palatable, accessible, affordable active agents for androgen suppression. And this provided an incentive to pursue part of the study subject of the instant invention.

For household animals such as dogs and cats, high doses of certain immidazole drugs, zinc gluconate, and zinc tannate have been used to achieve chemical castration, administered in injectable forms (U.S. Pat. No. 5,070,080; U.S. Pat. No. 7,276,535). Surprisingly, the prior art is silent on the use of these or similar drugs as components of palatable oral formulations. This prompted exploring part of the study, subject of the present invention, which deals not only with palatable oral formulations, but also their use in controlled release formulations to minimize the frequency of administration and extending the castration effect into virtually a permanent one. A logical extension of the chemical castration of household animals is the universal use of similar oral formulations for the chemical sterilization of the rapidly growing population of rodents, such as rats in large cities as well as squirrels and beavers mostly in agricultural and residential communities. And this provides a further incentive to explore the use of the oral formulation subject of the present invention for the chemical castration of those and other rodents.

### SUMMARY OF THE INVENTION

A major part of the invention deals with a palatable, solid, oral formulation for chemically sterilizing rodents such as rats, squirrels, and beavers comprising at least one bioactive agent, at least one edible additive, at least one processing aid, and at least one excipient, the bioactive agent selected from solasodine, solasodine salts, zinc arginine, ketoconazole, fluconazole, miconazole, gossypol, embellin, embellin derivatives, flutamide, zinc gluconate, zinc glycolate, zinc lactate, zinc sulfate, zinc alginate, zinc oxide, zinc tannate, an antiandrogen selected from cyproterone, cyproterone salts and ethinyl estradiols, and combinations thereof, wherein said formulation is in the form of beads, crushable or chewable spheroidal, ellipsoidal, or pill-like forms, and comprises a water-swellable polymer and wherein the at least one bioactive agent comprises (a) from about 0.01 to about 70 percent by weight of the total formulation; (b) zinc gluconate and wherein the zinc gluconate comprises from about 10 to about 60 percent by weight of the total formulation; (c) flutamide and wherein the flutamide comprises from about 0.1 to about 10 percent by weight of the total formulation; (d) miconazole nitrate and wherein the miconazole nitrate comprises from about 0.01 to about 5 percent by weight of the total formulation; (e) ketoconazole and wherein the ketoconazole comprises from about 0.01 to about 6 percent by weight of the total formulation; (f) a combination of from about 10 to about 60 percent by weight of zinc gluconate and from about 0.1 to about 10 percent by weight of flutamide; (g) is selected from embellin and an embellin salt and wherein the bioactive agent comprises from about 0.001 to about 4 percent by weight of the total formulation; and (h) is selected from gossypol and a gossypol complex with a carboxylic compound and wherein the bioactive agent comprises from about 0.001 to about 5 percent by weight of the total formulation.

A specific aspect of this invention deals with a palatable, solid, oral formulation for chemically sterilizing rodents such as rats, squirrels, and beavers comprising at least one bioactive agent, at least one edible additive, at least one processing aid, and at least one excipient, the bioactive agent selected from the group consisting of solasodine, solasodine salts, zinc arginine, ketoconazole, fluconazole, miconazole, gossypol, embellin, embellin derivatives, flutamide, zinc gluconate, zinc glycolate, zinc lactate, zinc sulfate, zinc alginate, zinc oxide, zinc tannate, an antiandrogen selected from cyproterone, cyproterone salts and ethinyl estradiols, and combinations thereof, wherein the at least one edible additive is selected from the group consisting of chicken bouillon, beef bouillon, dried milk, casein, dried eggs, butter-like flavor, and cheese flavor, and wherein the at least one processing aid is selected from the group consisting of calcium stearate, zinc state, calcium stearate, and magnesium stearate and further, wherein the at least one excipient is selected from the group consisting of bees wax, chitosan powder, soybean powder, and microcrystalline cellulose.

Another specific aspect of this invention deals with a palatable, solid, oral formulation for chemically sterilizing rodents such as rats, squirrels, and beavers comprising at least one bioactive agent, at least one edible additive, at least one processing aid, and at least one excipient, the bioactive agent selected from the group consisting of solasodine, solasodine salts, zinc arginine, ketoconazole, fluconazole, miconazole, gossypol, embellin, embellin derivatives, flutamide, zinc gluconate, zinc glycolate, zinc lactate, zinc sulfate, zinc alginate, zinc oxide, zinc tannate, an antiandrogen selected from cyproterone, cyproterone salts and ethinyl estradiols, and combinations thereof, wherein said formulation is made by the method comprising powder mixing all components and subsequently pressing the mixture, under pressure, in a mold having the required shape.

A second major part of this invention deals with a palatable, solid, oral, controlled drug release formulation for chemically sterilizing human and animal males comprising at least one bioactive agent ionically immobilized on a polymeric ion-exchanger, and mixed with at least one edible additive, at least one processing aid, and at least one excipient, wherein the formulation is shaped and has a polymeric enteric coating, the at least one bioactive agent selected from the group consisting of solasodine, solasodine salts, zinc arginine, zinc tannate, ketoconazole, fluconazole, miconazole, gossypol, embellin, embellin derivatives, flutamide, zinc gluconate, zinc glycolate, zinc lactate, zinc sulfate, zinc alginate, zinc oxide, an antiandrogen selected from cyproterone, cyproterone salts and ethinyl estradiols, and combinations thereof, wherein said formulation comprises (a) a water-soluble polymer; (b) polymeric ion-exchanger selected from an acrylic acid polymer or copolymer, an acrylic acid-sodium acrylate copolymer, acid-terminated polyglycolic acid microparticles, acid terminated polylactic acid microparticles, poly(glycolic-co-lactic acid) microparticles, C-succinylated polycaprolactone, and C-succinylated polyalkylene glycol; (c) the at least one edible additive is selected from the group consisting of sweetened coconut powder, chicken or beef bouillon, sweetened microcrystalline cellulose, and pulverized sucrose or glucose; (d) the at least one excipient is selected from the group consisting of pulverized corn starch and microcrystalline cellulose; (e) the at least one processing aid is selected from the group consisting of zinc stearate, magnesium stearate, and calcium stearate; and (f) the polymeric enteric coating is selected from the group consisting of hydroxypropyl cellulose, hydroxypropyl methyl cellulose, methyl cellulose, and hydroxypropyl methyl cellulose phthalate.

A key aspect of this invention deals with a palatable, solid, oral formulation for chemically sterilizing rodents such as rats, squirrels, and beavers comprising at least one bioactive agent, at least one edible additive, at least one processing aid, and at least one excipient, and the bioactive agent selected from the group consisting of solasodine, solasodine salts, zinc arginine, ketoconazole, fluconazole, miconazole, gossypol, embellin, embellin derivatives, flutamide, zinc gluconate, zinc glycolate, zinc lactate, zinc sulfate, zinc alginate, zinc oxide, zinc tannate, an antiandrogen selected from cyproterone, cyproterone salts and ethinyl estradiols, and combinations thereof, wherein said formulation comprises a water-swellable polymer selected from the group consisting of segmented polyether-ester, zinc alginate, and succinylated chitosan.

Another key aspect of this invention deals with a palatable, solid, oral, controlled drug release formulation for chemically sterilizing human and animal males comprising at least one bioactive agent ionically immobilized on a polymeric ion-exchanger, and mixed with at least one edible additive, at least one processing aid, and at least one excipient, wherein the formulation is shaped and has a polymeric enteric coating, the at least one bioactive agent selected from the group consisting of solasodine, solasodine salts, zinc arginine, zinc tannate, ketoconazole, fluconazole, miconazole, gossypol, embellin, embellin derivatives, flutamide, zinc gluconate, zinc glycolate, zinc lactate, zinc sulfate, zinc alginate, zinc oxide, an antiandrogen selected from cyproterone, cyproterone salts and ethinyl estradiols, and combinations thereof, wherein said formulation is made by the method comprising the steps of powder mixing all components and subsequently pressing the mixture, under pressure, in a mold having the required shape.

### DETAILED DESCRIPTION OF PREFERRED EMBODIMENTS

The present invention is generally directed to palatable, solid, oral formulations comprising at least one bioactive agent capable of the chemical sterilization or castration of human and animal males. Irrespective of the target male species, the compositions of all formulations are designed to be in ingestable solid forms such as beads, tablets, pills, or microparticles comprising at least one bioactive agent capable of affecting the sperm formation and/or viability, motility, and ability to effectively fertilize eggs of the subject female organism. All formulations comprise at least one edible additive, at least one processing aid, and at least one excipient. These compositions can also include a water-swellable polymer. However, the edible additives for targeted animal use are of the types that are conducive to those animals to ingest the composition through desirable odor or flavor perception. Formulations or compositions geared for animals are suitable to add to their regular dietary components or may be used as such. For both human and animal use, the formulations or bioactive compositions can be made to allow the prolonged release of the bioactive components and to extend the effective period of temporary castration. The frequency of administration of these compositions and prolonging their effectiveness can also result in a virtually permanent castration if so desired. For human use, a typical controlled release formulation can be made in the form of a pill or tablet with enteric coating to assist in controlling the release of the bioactive components. For both human and animals, the bioactive formulation can be in the form of swellable beads that undergo substantial size increase in the stomach environment before bursting and releasing the bioactive agent, thus prolonging the residence time and drug availability in the stomach and intestinal tract. The formulation ability to swell in the stomach environment can be effected by using as additives (1) super-absorbent polymers such as copolymers of acrylic and sodium acrylate; (2) amphiphilic block polymers comprising the water-soluble polyethylene glycol as blocks or segments in the polymer chain; (3) ionically crosslinked chitosan with polycarboxylic acid; (4) ionically crosslinked calcium or zinc alginate or similar carboxylic-acid-bearing polysaccharides; (5) ionic conjugates of basic active agents and carboxylic containing, water-soluble polymers such as succinylated chitosan and C-succinylated segmented polyether-ester such as those described in U.S. Pat. Nos. 7,005,420 and 7,138,464; or (6) a pill having a biodegradable amphiphilic enteric coating that swells and absorbs water to increase the effective size or volume of the pill or tablet and subsequently increases its residence time in the stomach, then slowly degrades leading to bursting and releasing the active agents. For both animal and human use, the bioactive agent(s) can be ionically immobilized on ion-exchanging micro- or nanoparticles forming ionic conjugates to allow their tunneling through the mucosal membrane of the digestive tract, thus increasing their biological effectiveness. Alternatively, the ionic conjugates, with or without a protective biodegradable coating, may be phogocytized in the intestinal tract, thus increasing their biological efficacy and prolonging their bioactivity. Typical ion-exchanging micro- or nanoparticles can be made of acid-terminated polyglycolide, poly-l-lactide, or copolymers of glycolide and l-lactide.

Among the novel features of this invention are those dealing with (1) the use of oral formulations for animal application as a preferred alternative to injectable formulations—this is particularly important when dealing with the chemical sterilization of certain rodents, such as rats, squirrels, and beavers where injection is impractical or practically impossible; (2) the use of controlled release formulations to extend the castration period and possibly achieving a practically permanent castration; (3) the use of drugs having well-established bioactivities that are totally unrelated to contraception by manifesting spermiostatic activity at least in humans—typical examples of these are the antifungal agents miconazole, fluconazole, and ketoconazole; and (4) the use of certain agents for animal castration that interfere with sperm formation without relying on injections and hence, minimizing toxicity—a typical example of these is gossypol.

Further illustrations of the present invention are provided by the following examples:

### EXAMPLE 1

### Preparation of a Typical Oral Formulation: General Method for Bioactive Pill Formation

For preparing a typical bioactive pill, the bioactive ingredients are thoroughly mixed with the edible components, additive(s), and excipient(s). The mixture is then pulverized and mixed thoroughly with the processing aid(s). The powder formulation is then pressed at room temperature into the desired form by applying a pressure of at least 5,000 lb. for 1 to 2 minutes.

### EXAMPLE 2

### Preparation of a Zinc Gluconate-Containing Pill

The preparation of a 1.5 g pill following the general method of Example 1 required the use of a stock formulation consisting of 52, 43, and 5 percent by weight of zinc gluconate, chicken bullion, and magnesium stearate, respectively. Using a pill press at 5,000 lb pressure, the mixed powder was pressed for about 2 minutes at room temperature to produce the desired pill.

### EXAMPLE 3

### Preparation of a Miconazole-Containing Pill

The preparation of a 1.5 g pill following the general method of Example 1 and pill pressing conditions of Example 2 required the use of a stock formulation consisting of about 7, 5, and 88 percent by weight of micronazole nitrate, magnesium stearate, and chicken bullion, respectively.

### EXAMPLE 4

### Preparation of a Miconazole/Zinc Gluconate-Containing Pill

The preparation of a 1.5 g. pill following general methods of Example 1 and pill pressing conditions of Example 2 required the use of a stock formulation consisting of about 7, 5, 52, and 46 percent by weight of miconazole nitrate, magnesium acetate, zinc gluconate, and chicken bullion, respectively.

### EXAMPLE 5

### Preparation of a Flutamide-Containing Pill

The preparation of a 1.5 g. pill following the general method of Example 1 and pill pressing conditions of Example 2 requires a stock formulation consisting of about 3, 5, and 92 percent by weight of flutamide, magnesium stearate, and chicken bullion of the total formulation, respectively.

### EXAMPLE 6

### Preparation and Characterization of an Acid-Terminated Poly-l-Lactide and Microparticles Thereof as Cation-Exchanger

A low molecular weight acid-terminated poly-l-lactide was prepared following the general procedure used for preparing acid-terminated polyglycolide as per U.S. Pat. No. 5,714,159, incorporated herein by reference, with the exception of using (1) malic acid as the initiator at a monomer/initiator molar ratio of 15; (2) a monomer/catalyst molar ratio of 10,000—stannous octanoate is the catalyst; and (3) a reaction temperature of 150° C. for a period of 3 hours. The resulting polymer was removed, micronized using a jet-mill, and rinsed with 2-propanol, filtered, and dried under reduced pressure at 25° C. then 50° C. until a constant weight was realized.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims. Moreover, Applicant hereby discloses all subranges of all ranges disclosed herein. These subranges are also useful in carrying out the present invention.

What is claimed is:

1. A palatable, solid, oral formulation for chemically sterilizing rodents such as rats, squirrels, and beavers comprising at least one bioactive agent, at least one edible additive, at least one processing aid, and at least one excipient, the bioactive agent selected from the group consisting of solasodine, solasodine salts, zinc arginine, ketoconazole, fluconazole, miconazole, gossypol, embellin, embellin derivatives, flutamide, zinc gluconate, zinc glycolate, zinc lactate, zinc sulfate, zinc alginate, zinc oxide, zinc tannate, and an antiandrogen selected from cyproterone, cyproterone salts and ethinyl estradiols, and combinations thereof.

2. A palatable, solid, oral formulation as in claim 1 comprising a water-swellable polymer.

3. A palatable, solid, oral formulation as in claim 1 in the form of beads, crushable or chewable spheroidal, ellipsoidal, or pill-like forms.

4. A palatable, solid, oral formulation as in claim 1 wherein the at least one bioactive agent comprises from about 0.01 to about 70 percent by weight of the total formulation.

5. A palatable, solid, oral formulation as in claim 4 wherein the at least one bioactive agent comprises zinc gluconate and wherein the zinc gluconate comprises from about 10 to about 60 percent by weight of the total formulation.

6. A palatable, solid, oral formulation as in claim 4 wherein the at least one bioactive agent comprises flutamide and wherein the flutamide comprises from about 0.1 to about 10 percent by weight of the total formulation.

7. A palatable, solid, oral formulation as in claim 4 wherein the at least one bioactive agent comprises miconazole nitrate and wherein the miconazole nitrate comprises from about 0.01 to about 5 percent by weight of the total formulation.

8. A palatable, solid, oral formulation as in claim 4 wherein the at least one bioactive agent comprises ketoconazole and wherein the ketoconazole comprises from about 0.01 to about 6 percent by weight of the total formulation.

9. A palatable, solid, oral formulation as in claim 4 wherein the at least one bioactive agent comprises a combination of from about 10 to about 60 percent by weight of zinc gluconate and from about 0.1 to about 10 percent by weight of flutamide.

10. A palatable, solid, oral formulation as in claim 4 wherein the at least one bioactive agent is selected from embellin and an embellin salt and wherein the bioactive agent comprises from about 0.001 to about 4 percent by weight of the total formulation.

11. A palatable, solid, oral formulation as in claim 4 wherein the at least one bioactive agent is selected from gossypol and a gossypol complex with a carboxylic compound and wherein the bioactive agent comprises from about 0.001 to about 5 percent by weight of the total formulation.

12. A palatable, solid, oral formulation as in claim 1 wherein the at least one edible additive is selected from the group consisting of chicken bouillon, beef bouillon, dried milk, casein, dried eggs, butter-like flavor, and cheese flavor.

13. A palatable, solid, oral formulation as in claim 1 wherein the at least one processing aid is selected from the group consisting of calcium stearate, zinc state, calcium stearate, and magnesium stearate.

14. A palatable, solid, oral formulation as in claim 1 wherein the at least one excipient is selected from the group consisting of bees wax, chitosan powder, soybean powder, and microcrystalline cellulose.

15. A palatable, solid, oral formulation as in claim 1 made by the method comprising powder mixing all components and subsequently pressing the mixture, under pressure, in a mold having the required shape.

16. A palatable, solid, oral, controlled drug release formulation for chemically sterilizing human and animal males comprising at least one bioactive agent ionically immobilized on a polymeric ion-exchanger, and mixed with at least one edible additive, at least one processing aid, and at least one excipient, wherein the formulation is shaped and has a polymeric enteric coating, the at least one bioactive agent selected from the group consisting of solasodine, solasodine salts, zinc arginine, zinc tannate, ketoconazole, fluconazole, miconazole, gossypol, embellin, embellin derivatives, flutamide, zinc gluconate, zinc glycolate, zinc lactate, zinc sulfate, zinc alginate, zinc oxide, and an antiandrogen selected from cyproterone, cyproterone salts and ethinyl estradiols, and combinations thereof.

17. A palatable, solid, oral formulation as in claim 16 further comprising a water-swellable polymer.

18. A palatable, solid, oral, controlled drug release formulation as in claim 16 wherein the polymeric ion-exchanger is selected from the group consisting of an acrylic acid polymer or copolymer, an acrylic acid-sodium acrylate copolymer, acid-terminated polyglycolic acid microparticles, acid terminated polylactic acid microparticles, poly(glycolic-co-lactic acid) microparticles, C-succinylated polycaprolactone, and C-succinylated polyalkylene glycol.

19. A palatable, solid, oral, controlled drug release formulation as in claim 16 wherein the at least one edible additive is selected from the group consisting of sweetened coconut powder, chicken or beef bouillon, sweetened microcrystalline cellulose, and pulverized sucrose or glucose.

20. A palatable, solid, oral, controlled drug release formulation as in claim 16 wherein the at least one excipient is selected from the group consisting of pulverized corn starch and microcrystalline cellulose.

21. A palatable, solid, oral, controlled drug release formulation as in claim 16 wherein the at least one processing aid is selected from the group consisting of zinc stearate, magnesium stearate, and calcium stearate.

22. A palatable, solid, oral, controlled drug release formulation as in claim 16 wherein the polymeric enteric coating is selected from the group consisting of hydroxypropyl cellulose, hydroxypropyl methyl cellulose, methyl cellulose, and hydroxypropyl methyl cellulose phthalate.

23. A palatable, solid, oral, controlled drug release formulation as in claim 1 comprising a water-swellable polymer selected from the group consisting of segmented polyether-ester, zinc alginate, and succinylated chitosan.

24. A palatable, solid, oral, controlled drug release formulation as in claim 16 made by the method comprising the steps of powder mixing all components and subsequently pressing the mixture, under pressure, in a mold having the required shape.
